(19) United States
(12) Patent Application Publication
(10) Pub. No.: US 2025/0057908 A1
(43) Pub. Date: Feb. 20, 2025

(54) SUPPLEMENT FORMULA AND RELATED METHODS

(71) Applicant: Me Biosciences, Inc., Boulder, CO (US)

(72) Inventors: Murdoc Khaleghi, Denver, CO (US); Jean Paul O'Brien, Boulder, CO (US); Connor Nisbet, Wilmington, DE (US); Barry Luginbill, Erie, CO (US); Aaron Beach, Erie, CO (US); Eric McCarty, Boulder, CO (US); Rob Hennen, Provo, UT (US); Alec Weir, Saginaw, MI (US)

(21) Appl. No.: 18/720,897

(22) PCT Filed: Jan. 13, 2023
(86) PCT No.: PCT/US23/10790

§ 371 Date: Jun. 17, 2024

Related U.S. Application Data
(60) Provisional application No. 63/299,461, filed on Jan. 14, 2022.

(51) Int. Cl. A61K 36/84; A23L 33/105; A23L 33/155; A23L 33/16; A23L 33/175; A61K 31/198; A61K 31/592; A61K 31/593; A61K 33/06; A61K 36/53; A61K 36/736; A61K 36/81

(52) U.S. Cl. CPC A61K 36/84; A23L 33/105; A23L 33/155; A23L 33/16; A23L 33/175; A61K 31/198; A61K 31/592; A61K 31/593; A61K 33/06; A61K 36/53; A61K 36/736; A61K 36/81

(57) ABSTRACT

A method of improving patient recovery after a surgery includes: providing a pre-operative protocol, the pre-operative protocol having a first set of instructions for the consumption of a first nutritional supplement; and providing a post-operative protocol, the post-operative protocol having a second set of instructions for the consumption of a second nutritional supplement. The first set of instructions has an instruction to consume or administer the pre-operative protocol for seven days prior to the surgery. The first nutritional supplement has a base, vitamin D, Calcium, magnesium, potassium, sodium, ashwagandha, glutamine, and arginine in quantities selected to improve recovery after surgery. The second set of instructions has an instruction to consume or administer the post-operative protocol for thirty days after surgery. The second nutritional supplement has a base, vitamin D, Calcium, magnesium, potassium, sodium, ashwagandha, glutamine, and arginine in quantities selected to improve recovery after surgery.

### CROSS-REFERENCE TO RELATED APPLICATIONS

This application claims priority to U.S. Provisional Application No. 63/299,461, filed Jan. 14, 2022 and entitled “SUPPLEMENT FORMULA AND RELATED METHODS,” the entire disclosure of which is hereby incorporated by reference for all proper purposes.

### FIELD

This invention is related to nutritional supplements. Specifically, but not intended to limit the invention, embodiments of the invention are related to nutritional supplements and methods for improving patients' health.

### BACKGROUND

A number of nutritional supplements, pharmaceutical products, and food sources are known to affect the quality and health of the population. There remains a need, however, for a product that provides synergistic results for improving general health, recovery from surgical procedures, and/or improved sleep, and/or other new and useful products and methods.

### SUMMARY

An exemplary method of improving patient recovery after a surgery includes: providing a pre-operative protocol, the pre-operative protocol having a first set of instructions for the consumption of a first nutritional supplement; and providing a post-operative protocol, the post-operative protocol having a second set of instructions for the consumption of a second nutritional supplement different from the first nutritional supplement. The first set of instructions includes an instruction to at least one of consume or administer the pre-operative protocol for seven days prior to the surgery. The first nutritional supplement includes a base, vitamin D, Calcium, magnesium, potassium, sodium, ashwagandha, glutamine, and arginine in quantities selected to improve recovery after surgery. The second set of instructions includes an instruction to at least one of consume or administer the post-operative protocol for thirty days after surgery. The second nutritional supplement includes a base, vitamin D, Calcium, magnesium, potassium, sodium, ashwagandha, glutamine, and arginine in quantities selected to improve recovery after surgery.

An exemplary perioperative protocol, includes: a pre-operative protocol, the pre-operative protocol having a first set of instructions for the consumption of a first nutritional supplement; and a post-operative protocol, the post-operative protocol having a second set of instructions for the consumption of a second nutritional supplement different from the first nutritional supplement. The first set of instructions has an instruction to at least one of consume or administer the pre-operative protocol for seven days prior to the surgery. The first nutritional supplement has a base, vitamin D, Calcium, magnesium, potassium, sodium, ashwagandha, glutamine, and arginine in quantities selected to improve recovery after surgery. The second set of instructions has an instruction to at least one of consume or administer the post-operative protocol for thirty days after surgery. The second nutritional supplement has a base, vitamin D, Calcium, magnesium, potassium, sodium, ashwagandha, glutamine, and arginine in quantities selected to improve recovery after surgery.

### BRIEF DESCRIPTION ON THE DRAWINGS

FIG. 1 is a flowchart of a method; and

FIG. 2 is a flowchart of a protocol suitable for use in the method of FIG. 1.

### DETAILED DESCRIPTION

Surgery is taxing on the mind and body, and many struggle to sleep leading up to surgery, and nearly always after surgery. In addition, the stress and anxiety pre and post operation takes a toll on one's mind and body. It has only recently been discovered, however, that one's nutrition is supremely important in preparing patients for the physical and mental stress of surgery and/or improving recover afterwards. To provide improved surgery response, Applicant has developed a supplement(s) and method, as disclosed herein.

In some embodiments, a pre- and post-surgery protocol includes of two daily drinks and OMEGA's ensures the patient enters surgery in optimal condition

In some embodiments, a post-surgical protocol is provided to optimize the traditional post-surgery recommendations (i.e. physical therapy, to speed recovery).

While many have published nutritional recommendations for general health, Applicant has targeted specific research to expedite post-surgical recovery. Applicant used the Enhanced Recovery After Surgery (ERAS) protocol as a starting point, and further developed, expanded and optimized the nutritional aspects of the ERAS, researching both niche and general aspects of nutrition in recovery. Novel embodiments disclosed herein reduce recovery time and muscle catabolism, improve sleep, minimize pain and excess inflammation, reduce opioid usage, improve digestion, normalize endocrine and hormone function, and reduce post-surgery complications.

Embodiments herein may include a pre-operative supplement formula and 7-day, 1 to 7-day, 7 to 14-day, and 14 to 21-day pre-operative protocols.

Embodiments herein may include a post-operative supplement formula and 4, 8, 1 to 4, 4 to 12, or 12-26-week post-operative protocols.

Embodiments herein may include an omega supplement formula and a 4, 8, 1 to 4, 4 to 12, or 12-20-day omega protocols.

Embodiments herein may include a sleep supplement formula and 4, 8, 1 to 4, 4 to 12, or 12-26-week sleep protocol.

Meeting the caloric needs of each patient is critical to recovery and reduction of postoperative complications. Depending on the surgical group, 8-68% of patients will enter the operating room malnourished. Ensuring proper calorie balance after surgery, initiating postoperative feeding as soon as possible, and entering surgery sufficiently nourished all play significant roles.

Patients are advised to weight themselves before surgery and to continue to monitor and manage weight throughout the recovery process. Losing significant weight should be extremely concerning in terms of long term health, recovery, diminishing mortality rates, and preventing infection. Avoiding weight loss should perhaps be the primary goal of this nutrition program.

Losing more than 10% body weight is correlated significantly with postoperative complications, with more than 5% body weight loss over a 1-month period considered a clinical indicator of malnutrition, especially in the context of chronic inflammation.

A diet composition comprised largely of protein and fats (approximately a 2:1 ratio of carbohydrates to protein, or carbohydrates making up 40% of one's recovery diet) is most conducive towards body composition, and hormone production, especially in older patients. Specific diet composition is somewhat controversial, as different studies have emphasized carbohydrates or fats more than others.

Nonetheless, the most important consensus takeaway is that meeting and exceeding energy expenditure, along with supplementing sufficient protein/amino acids is crucial. With that, quality protein should be included in patients' meals; for example, some embodiments herein include a meal or a meal replacement having at least 5 grams, or between 5 and 45 grams, or between 5 and 25 grams, or between 25 30 grams, or between 30 and 45 grams of quality protein therein.

There are various indirect methods of calculating energy demand and caloric expenditure. Generally, the formula takes in resting metabolic rate (RMR), activity level, and stress factor:

$$Energy Expenditure = RMR * Activity Level * Stress Factor$$

While maintaining enough calories is important for every surgery patient, preventing adipose weight gain as a result of over prescription of calories is especially important in orthopedic patients who wish to return to full performance as soon as possible. Using judgement and advice from dietitians and physicians on specific calorie intake and which way to err is also an important factor.

### Caloric Resting/Basal Metabolic Rate Calculations

The competing methodologies for calculating RMR (Resting Metabolic Rate)/BMR (Basal Metabolic Rate) include the Harris and Benedict Equations (HBE) and Mifflin-St Jeor Equations, with the HBE generally used as the gold standard. The equations differ between men and women.

### Harris and Benedict Equations (HBE):

$$Women \text{:} BMR = 655 + ( 4.35 * weight ) + ( 4.7 * height ) - ( 4.7 * age )$$

$$Men \text{:} BMR = 66 + ( 6.23 * weight ) + ( 12.7 * height ) - ( 6.8 * age )$$

### Mifflin-St Jeor Equations:

$$Male \text{:} BMR = ( 10 * weight ) + ( 6.25 * height ) - ( 5 * age ) + 5$$

$$Female \text{:} BMR = ( 10 * weight ) + ( 6.25 * height ) - ( 5 * age ) - 161$$

More recently, however, it is understood that the metabolic process is not simply a matter of calculating activity and desired weight. For example, the body goes through increased muscle catabolism, anabolism, tissue healing, and sometimes immune or inflammatory responses as a result of surgery or illness, and, as a result, a patient's caloric demands may increase. This increase in caloric demand is called the stress factor, and its magnitude depends on the sustained injury. Table 1 below is a table laying out the stress factor depending on the injury and body's state.

TABLE 1

| Stress Factor | Stress Insult |
|---|---|
| Minor Injury | 1.2 |
| Minor Surgery | 1.2 |
| Clean Wound | 1.2 |
| Bone fracture | 1.5 |
| Infected Wound | 1.5 |
| Major Trauma | 1.5 |
| Severe Burn | 1.5 |

Finally, to complete the calculations for meeting caloric needs, one should factor in the activity level of the patient. While this is done on a more consistent basis through wearable technology some situations may produce unreliable results. Using the following activity level formula shown in Table 2 in these instances may be beneficial.

TABLE 2

| Activity | Activity Level |
|---|---|
| Bed Ridden | 1 |
| Sedentary | 1.2 |
| Light Active | 1.375 |
| Moderately Active | 1.55 |
| Very Active | 1.725 |
| Extra Active | 1.9 |

Minimizing the pre- and post-operative fasting period is also important to maintaining proper nutrition, preventing insulin resistance, and improving appetite, sleep, and mental state. Research suggests that solid food can be consumed safely 6 hours before surgery, with clear liquids safe up to 2 hours before surgery. To capitalize on this, a preoperative carbohydrate drink will increase calorie consumption the day before surgery, along with increasing circulating glucose levels, which decreases glucagon release due to stress and thereby decreasing insulin resistance.

After surgery, PO (poros, or by mouth) postoperative feeding should start as soon as possible. This works both to resume regular digestion and to start re-fueling calories and protein. If feeding is not possible right after surgery, chewing gum will help to restart regular digestion and gastrointestinal function, and is therefore recommended.

After surgery, the body goes through a period of extreme catabolism, especially when in a state of nitrogen or caloric deficiency. To combat this, it is advisable to meet both caloric and amino acid requirements (especially in the first few days after surgery, when nitrogen loss is especially high). Surgery recovery requires specific amino acid supplementation.

In some embodiments, essential amino acids are the only amino acids needed to maintain nitrogen balance and promote muscle anabolism. Essential amino acids include histidine, isoleucine, leucine, lysine, methionine, phenylalanine, threonine, tryptophan and valine. In times of extremely muscle catabolism, it is critical to include increased levels of essential amino acids in the patient's diet. Of these leucine (or β-Hydroxy-β-methylbutyrate), isoleucine and valine are critical.

Leucine, a branched chain amino acid, and its metabolite β-Hydroxy-β-methylbutyrate (HMB) play roles as a substrate for protein synthesis and to activate enzymes within the mammalian target of rapamycin (mTOR) signaling pathway. Some embodiments herein include a post-surgery recovery formula having up to 4.5 grams, between 1 and 2.5 grams, or between 2.5 and 3 grams, or between 3 and 4.5 grams of leucine. It is noted, however, that some studies have shown that HMB can be a more effective mode of leucine supplementation. As a metabolite of leucine, it would take 60 g of leucine supplementation to produce 3 g of leucine.

Other branched chain amino acids (BCAAs) include valine and isoleucine, and have been shown to attenuate soreness and increased recovery, including increased nitrogen retention. Note, however, despite this increased nitrogen retention, there are discrepancies on whether there was a significant increase in body weight versus the placebo, non-essential amino acid group. More research may be needed to reinforce the necessity of BCAAs, but, as there is a general consensus for the necessity of essential amino acids in general, there seems to be only upside in supplementing BCAAs.

Conditionally essential amino acids include arginine, cysteine, glutamine, glycine, proline, serine, and tyrosine. Conditionally essential amino acids are generally non-essential, unless the body is under high stress, such as after undergoing surgery. Some specific conditionally essential amino acids have been shown to be beneficial in preventing muscle catabolism and promoting tissue and specifically muscle repair and anabolism. These include arginine, glycine, and glutamine.

Glutamine, in L-alanyl-L-glutamine form, can additionally aid in fluid and electrolyte balance along with nitrogen retention.

One of the most common complaints after surgery is excessive inflammation. While some levels of inflammation after tissue damage signifies a functioning immune system, excessive inflammation can lead to pain and tissue damage, along with a variety of diseases. Embodiments described herein provide nutritional remedies for inflammation (and subsequently pain), and/or reduce the use of opioid or non-steroidal anti-inflammatory drugs (NSAIDs), which can exacerbate problems during recovery.

One of the most common natural ingredients to reduce both chronic and acute inflammation is omega-3s (n-3s). These polyunsaturated fats decrease the production of inflammatory eicosanoids and cytokines. There are three general types of omega-3s, alphalinolenic acid (ALA), eicosapentaenoic acid (EPA), and docosahexaenoic acid (DHA). While there is some minor discrepancy in the recommended ratios of each omega-3 to take, studies almost uniformly agree that consumption of omega-3s can decrease inflammation, shown through decreased hsCRP levels, decreasing incidence of strokes and myocardial infarction, improving amino acid uptake, and improved recovery, all without increases in operative bleeding.

In some embodiments, a mix of DHA and EPA is provided, at a ratio of about 2 parts DHA to 1 part EPA.

Cellular respiration—critical to maintaining life—also comes with the by-product of oxidized free radicals. These free radicals have a variety of both positive and negative roles in the body, including stimulating the immune system against antigens, and negative effects like lipid and DNA oxidation. The negative effects of these free radicals (called oxidative stress) is modulated by our system of antioxidants, which enzymatic antioxidants, which some nutrients contribute to the synthesis of, and non-enzymatic antioxidants, that include vitamin E, vitamin A, vitamin C, glutathione and uric acid, and come straight from our diet. An imbalance in oxidants and antioxidants leads to high levels of oxidation in the body, which can lead to cell apoptosis, decreased cognitive function and inflammation. Oxidative stress is increased after surgery, and therefore we must bolster our bodies antioxidant reserves before/after surgery.

There are a variety of natural antioxidants that have been studied on different benchmarks. Green tea extract is a commonly used and studied antioxidant. Green tea is full of polyphenols, or more specifically flavonols, which have strong antioxidant qualities. These flavonoids have been shown to reduce inflammation in diabetics, improve gut quality, which improves our sleep, mood through the endocrine system, decreases oxidation in athletes after exercise, and improves outcomes in surgical patients, all of which is probably largely a result of the antioxidation qualities of green tea.

Vitamin E (specifically α-tocopherol (AT)), much like polyphenols in green tea extract, is a non-enzymatic antioxidant. Vitamin E is important in attenuating lipid peroxidation, and vitamin E deficiency—a common occurrence—can exacerbate stress injuries. Vitamin E has been shown to decrease oxidative stress and inflammation, along with improving mortality in different patient groups, including cardiovascular disease (CVD) patients, attenuates oxidative stress-induced cognitive decline, sports recovery, hyperthyroidism, and surgery recovery. While there is some controversy on the effectiveness of Vitamin E supplementation, for almost all patients, Vitamin E and general antioxidant consumption is safe and probably effective.

Note: Cancer patients, specifically breast cancer patients, should be wary of antioxidant intake, as it can interfere with chemotherapy. This is expanded further in other sections of this document.

Vitamin C is a common antioxidant that has also shown promise in tendon and bone healing. A critical ingredient in collagen synthesis, optimal immune response, cell mitosis, and cell differentiation. As 25% of non-smoking men and 50% of smoking men are deficient in vitamin C, supplementation may be important simply to achieve sufficient vitamin C. Some embodiments herein include a supplement having between 100 and 300 milligrams, or between 300 and 500 milligrams, or between 500 and 700 milligrams of vitamin C.

Pain management might be the most important side effect of surgery recovery to manage. Pain is obviously uncomfortable and decreases quality of life in patients, with prolonged and/or severe bouts of pain constituting an emergency. Additionally, avoiding the use of strong painkillers, like opioids, can be both beneficial to reducing endocrine response to surgery, improving digestion after surgery, and avoiding long-term dependence on opioids. A large portion of managing pain comes with managing inflammation, which is described in other sections of this document.

Patient education can be critical to calming patients' fears before surgery, ensuring confidence, and developing a pain plan (specifically to maximize comfort and minimize opioid usage). The patient should know the pros, cons, risks, and benefits of every option they choose, and what to expect after the surgery.

Curcumin, an extract from cinnamon, is another strong natural anti-inflammatory and antioxidant. As an anti-inflammatory, curcumin also works to manage pain through inhibiting the COX-1 and COX-2 genes, a key player in inflammation. Curcumin can also combat high glucose levels after surgery that can be a result of surgical stress response or insulin resistance.

Perioperative anxiety is both normal and detrimental to surgery recovery, and managing anxiety should be a part of the postoperative plan. ACTH (adreno-corticotropic hormone) is released along with a variety of other hormones, stimulating glucocorticoid, and more specifically, cortisol release. Cortisol has a variety of negative effects on the body, including suppressing appetite, blood pressure and cholesterol levels, and general, overall health.

As stress also affects sleep time, sleep quality, and particularly the time it takes to fall asleep, many of these anti-anxiety ingredients will have beneficial effects on sleep and may also be included in sleep formulas.

Ashwagandha root mediates stress through active ingredients of with anolides, sitoindosides and other alkaloids, which also work to protect the body from oxidative stress. Administration of 300 mg of Ashwagandha root over 10 weeks was shown to improve sleep latency and efficiency in anxious insomniacs, 250-600 mg reduced stress in stressed patients, and reduced cortisol levels with dosages of 300 mg twice daily. While more specific research needs to be done with Ashwagandha root in surgical patients, all evidence points to efficacy of Ashwagandha root in treatment of stress, anxiety, and high cortisol levels, both before and after surgery.

Lysine and arginine are two essential amino acids. Arginine can be beneficial for a variety of reasons, namely reducing catabolism by improving post-surgery nutrition and overall health. Some embodiments herein include a supplement having between 1 and 2 grams, or between 2.0 and 2.64 grams, or between 2.64 and 3 grams of lysine. Some embodiments herein include a supplement having between 1 and 2 grams, or between 2.0 and 2.64 grams, or between 2.64 and 3 grams of arginine (L-lysine and L-arginine). Some embodiments herein include lysine and arginine in levels selected to modulate a patient's hormonal response to stress, reducing salivary cortisol and chromogranin-A (a salivary marker in the sympathoadrenal system). These effects are believed to be modulated dually through lysine's role as a serotonin receptor 4 antagonist, and benzodiazepine agonist. These results were duplicated in a study on transported pigs (significantly reduced cortisol and chromogranin-A levels), but, as it was a study on non-humans, these results are much less meaningful than the results from Smriga's Oral treatment with L-lysine and L-arginine reduces anxiety and basal cortisol levels in healthy humans.

In some embodiments, amino acids like L-tryptophan and/or L-tyrosine may be provided to treat high cortisol and anxiety levels.

Perhaps the most important aspect of recovery is sleep. Sleep is one of the most important predictors and factors in early mortality, anxiety, cardiovascular disease, quality of life, stress, memory building, and tissue recovery. Reducing sleep latency, improving sleep consistency, and decreasing sleep disturbances all improve overall sleep quality. Sleep disturbances can be exacerbated in hospital settings, as a hospital is generally filled with bright lights, loud noises, and frequent disruptions, making capitalizing on what sleep time the patient can get especially important. Many pain and anxiety reducing ingredients will also be productive in attaining better sleep, along with adherence to points in psychological considerations.

Valerian is the common name given to the genus Valeriana, herbaceous perennial plants widely distributed in the temperate regions of North American, Europe and Asia. Valerenic acid has been found to inhibit the breakdown of GABA (gamma-Aminobutyric acid—a simulator neurotransmitter) in the brain, resulting in feelings of calmness and tranquility. Valerian root also contains the antioxidants hesperidin and linarin, which appear to have sedative and sleep-enhancing properties. The valerian extract resulted in a statistically significant subjective improvement in time required to fall asleep (more or less difficult than usual), sleep quality (better or worse than usual), and number of nighttime awakenings (more or less than usual). These improvements were seen almost exclusively in patients with poor baseline sleep, and studies differ on whether valerian root improves the sleep of normal sleepers. Research recommends taking 300-600 mg of valerian root 2 hours before bed for optimal sleep improvements. While most studies have shown benefits of valerian root on sleep, there has also been some publication bias on valerian root studies, which warrants some further research on this subject to certify the results of previous studies.

Tart cherries (Prunus cerasus L) are widely used sleep remedies. Not only has tart cherry juice been shown to have significant benefits in insomniac patients, it also importantly lacks the harmful side effects of melatonin supplements, or benzodiazepines. Important in understanding why tart cherry juice is effective, and how it works is understanding what melatonin is, and how it is produced in the body. Melatonin is a hormone important in managing our circadian rhythms and is produced in the presence of darkness. Partially because of this, melatonin levels peak around 12-3 am. Higher levels of melatonin directly increase sleepiness. Melatonin supplements artificially increase circulating melatonin levels, which subsequently allow for a faster, longer sleep.

Artificial melatonin supplementation, however, has shown some minor side effects, including headaches and drowsiness.

Unlike melatonin, tart cherries work both through some levels of endogenous melatonin and through improving our gut microbiota. Melatonin is synthesized from the degradation of tryptophan (an essential amino acid) along one of its two major pathways in our gut microbiome—kynurenine pathway and serotonergic pathway—the later producing melatonin and serotonin, with the former producing mainly toxins. Further than just melatonin, the gut microbiota regulates sleep through the neuroendocrine pathway, using the hypothalamic-pituitary-adrenal axis (HPA axis) and central nervous system (CNS) to regulate the secretion of neurotransmitters such as cortisol, tryptophan, and serotonin. Additionally, studies have shown that insomnia patients have differing gut microbiota compositions than that of normal sleepers.

Tart cherry juice is effective in improving sleep quality because it acts as a prebiotic, positively influencing gut microbiota and pushing the metabolism towards the serotonergic pathway and improving signals sent through the neuroendocrine pathway.

The important active ingredient in tart cherries are the polyphenols, and more specifically anthocyanins (or flavoids). These anthocyanins have a pre-biotic effect, which is what causes the increased health in gut flora mentioned herein.

Tart cherries are also popular for their antioxidant and anti-inflammatory qualities. While it is well known that good sleep reduces inflammation and oxidative stress, it is unclear if lower oxidative stress and inflammation also promotes better sleep.

Some embodiments herein include a supplement having about 500 milligrams, or at least 500 milligrams, or between 200 and 1,000 milligrams, or between 500 and 800 milligrams of tart cherry juice, selected at levels to improve sleep quality, especially in habitually poor sleepers, and those with disrupted gut microbiomes.

In some cases, especially right after surgery, sleep and natural circadian rhythms can be disrupted to a degree that somewhat necessitates the use of melatonin. 5-6 mg of melatonin can be tolerated well and without significant side effects, all while having significant benefits on sleep. Timing of melatonin supplementation can be important as melatonin taken too early can promote drowsiness before sleep, while excess melatonin supplementation can lead to excess drowsiness in the morning.

Surgery, and the subsequent stress response, can have detrimental effects on our immune system. Depending on the patient, this can have varying con-sequences, including deteriorating our gut microbiome and opening our body up to infections. Embodiments herein, including those having omega-3s, proper nourishment (combating malnutrition), and synbiotic intake decreases incidences of infection. Because of this, supplementation with synbiotics (fibre and Lactobacillus), omega-3s (following inflammation protocol) and arginine is recommended, especially for patients with a suppressed immune system and patients at risk of malnutrition.

Digestion (gastrointestinal function) can be altered after surgery for various reasons, including stress response, surgery to an organ in the GI tract, or long bouts of NPO. Resumption of normal gastrointestinal function is critical in determining morbidity and patient stay in GI surgeries, but also plays a significant role in patient recovery and stay in almost every surgery.

If a patient is NPO after surgery—which stands for nil per os—means that a patient cannot intake any food or drink by mouth. This can occur for a variety of reasons, either in preparation for further tests, or because of injury to the GI tract. If a patient is NPO for an extensive time, sham feeding, for example chewing without swallowing (chewing gum usually), can help restart normal gastric function including cephalic-vagal stimulation of digestion and enhanced duodenal alkaline secretion.

Fluid balance is an important factor in both overall health, surgery recovery and maintaining general gastrointestinal (GI) function. Both overhydration and underhydration, along with too many or too few electrolytes can cause recovery problems, excess weight gain, delayed return to normal GI function, and extended hospital stay. Carefully calculating salt and fluid intake decreases chances of postoperative complications. Overhydration is more relevant and problematic in healthier patients, and underhydration is more relevant and problematic for critically ill patients.

Electrolyte levels should be monitored and supplemented in surgical patients. Patients require approximately 2-4 mmol/kg/day of potassium, approximately 0.3-0.6 mmol/kg/day of phosphate and approximately 0.2 mmol/kg/day or magnesium if supplied intravenously or 0.4 mmol/kg/day if supplied orally. Letting electrolyte levels drop substantially leaves patients susceptible to cardiac electrical issues and decreased renal function.

Most surgery recommendations should stay fairly consistent across all demographics or surgery types. Some groups face specific, specialized problems that need to be addressed or avoided, depending on the patient's needs. Cancer patients have various needs that may call for alternative embodiments of the invention.

Weight management should be part of every surgery recovery protocol, per ERAS, however, the importance of weight management should be stressed in cancer patients, as wasting is of particular concern in cancer patients principally in chemotherapy and radiation therapy, as diet can be suppressed in these patients. Weighing oneself before, after, and periodically during recovery can help determine if the surgery protocol is working, or if changes need to be made. As eating is suppressed in chemotherapy and radiation therapy patients, setting reminders to eat can be important to ensuring adequate/optimal intake.

Anti-inflammatories can be beneficial in attenuating post-surgical pain, inflammation, and various other side effects. Excessive anti-inflammatory intake, specifically in omega-3s, can diminish a patient's immune system. This can pose serious risks for immunocompromised patients, which include patients undergoing chemotherapy and radiation therapy. That said, omega-3s have real upsides, as mentioned earlier, especially in fighting cachexia in cancer patients. Starting omega-3 supplementation early (5-10 weeks before surgery) and at lower levels is optimal for recovery.

Cancer patients—particularly chemotherapy patients-should be wary and probably eliminate use of antioxidants. Some oncologists believe that antioxidant supplementation can interfere with chemotherapy treatments, and some antioxidants have been shown to worsen outcomes. Ensuring proper dietary vitamin C, magnesium, and calcium may improve outcomes, though there is no need to supplement these vitamins and minerals if sufficient levels are taken through the patient's diet.

Cancer treatments, along with surgical stress, are harmful to our gut. This can contribute to a myriad of side effects, including pelvic radiation disease, including mucositis, diarrhea, systemic inflammatory response, and pelvic radiotherapy-associated fatigue. As synbiotics (both pro and prebiotics) have been shown to be safe in cancer patients, supplementing synbiotics can help attenuate dysbiosis side effects and repair intestinal microbiota structure.

Recovering from surgery can be not only physically demanding, but psychologically demanding as well. Taking psychological considerations not only improves patient comfort, it can have real effects in physical recovery as well. While many of these suggestions may be impractical or outside the scope of our product, it is still worth noting and is part of the ERAS protocol.

Additionally, positive reinforcement during rehab has been shown to improve patient psyche and recovery.

As mentioned before, formulating a pain plan with the patient can help decrease the patient's usage of benzodiazepines and opioids. Additionally, psychological preparation before surgery can decrease recovery and hospital stay time.

Something as seemingly trivial as listening to music before, during and/or after surgery may seem pointless, but research has shown that it can calm the patient and decrease cortisol levels. With the wide availability and little to no cost for implementation, there seems little to no downside in employing music therapy.

Some research suggests that dietary antioxidants, including coffee, can improve cognitive function and decrease suicidal urges after surgery. This is particularly important in older adults, as they can experience cognitive dysfunction for days, weeks, or even months after surgery.

For broken bones, along with ensuring proper vitamin C intake, antioxidants, and caloric intake (see caloric guide), 1 g of calcium and 800-2000 IUs of vitamin D supplementation may be necessary to promote calcium uptake and absorption. 50 mg of zinc promotes osteoblast differentiation, and for growth and development of bone cells. With that, ensuring proper vitamin A intake (both too high and too low levels of vitamin A intake are associated with decreased bone mineral density (BMD)) can promote faster recovery. The proposed safe upper limit for vitamin A supplementation is 10,000 IUs, but most people do not need nearly that much.

The research is clear that implementation of good nutrition for postoperative recovery can expedite recovery, improve sleep, decrease stress, help manage pain, and attenuate side effects. The Enhanced Recovery After Surgery protocol provides a great starting point for surgical recovery, but this review, and further research, was and is needed to curtail specific nutritional approaches to recovery.

There are known relationships between inadequate sleep and a wide range of disorders, including hypertension, obesity and type-2 diabetes, impaired immune functioning, cardiovascular disease and arrhythmias, mood disorders, neurodegeneration and dementia, and even loneliness. In the United States 1 in 3 adults do not get enough sleep on a consistent basis, and Lack of Sleep costs the United States over $411 Billion Annually.

Prescription sleep aids are available; however, they are known for having significant adverse effects. There are options that on the other hand work to supplement the body's natural circadian rhythm to support regular, high quality sleep.

Embodiments of the invention described below may be intended for adults (age 18 or greater).

Embodiments may include a nutritional supplement having tart cherry. Tart cherry increases endogenous levels of melatonin, which affects the body's sleep-wake cycle. The sleep/wake cycle influences the nocturnal body temperature, which helps facilitate sleep readiness.

Embodiments may include a nutritional supplement having valerian root, such as at least 500 milligrams, or about 500 milligrams, or between 200 and 1,000 milligrams, or between 500 and 800 milligrams of valerian root. In some embodiments, a patient is instructed to consume the supplement approximately 2 hours before bed.

Valerenic acid has been found to inhibit the breakdown of GABA in the brain, resulting in feelings of calmness and tranquility. Valerian root also contains the antioxidants hesperidin and linarin, which appear to have sedative and sleep-enhancing properties. The valerian extract results in a statistically significant subjective improvement in time required to fall asleep (more or less difficult than usual), sleep quality (better or worse than usual), and number of nighttime awakenings (more or less than usual).

Embodiments of a nutritional supplement herein may include magnesium glycinate, such as at least 64 milligrams, or about 64 milligrams, or between 64 and 200 milligrams of magnesium glycinate per dose. Some embodiments may include instructions to take the supplement with food, and together with a vitamin B complex supplement to improve absorption.

Magnesium is an essential mineral, one of seven essential macro-minerals that the human body needs in large quantities. The body does not produce magnesium. The magnesium the body needs must come from outside sources. Magnesium glycinate is used because it has a calming effect on the brain due to the presence of glycine. It can help relieve anxiety and promote better sleep.

Embodiments herein may include L-theanine, such as at least 250 milligrams, or about 250 milligrams, or between 250 and 400 milligrams of L-theanine per dose, or daily in some embodiments.

L-theanine elevates levels of GABA, as well as serotonin and dopamine, neurochemicals that regulate emotions, mood, concentration, alertness, and sleep, as well as appetite, energy, and other cognitive skills. At the same time, L-theanine also reduces levels of chemicals in the brain that are linked to stress and anxiety. L-theanine boosts production of alpha waves in the brain, which enhance relaxation and focus. It helps people relax before bedtime, get to sleep more easily, and sleep more deeply.

Embodiments herein may include a protocol including the use of lavender as an aroma therapy and/or nutritional supplement.

Lavender may improve sleep quality, including in people with insomnia, depression, and anxiety. Lavender scent increases slow-wave sleep, instrumental for slowing heartbeat and relaxing muscles. Subjects have also been found to sleep more soundly on a lavender night. Lavender oil is suggested to modulate GABAergic neurotransmission, especially on GABAA receptors, and enhance inhibitory tone of the nervous system.

Embodiments of exemplary protocols and ingredients are shown in Tables 3-7 below. The protocols in Tables 3-7 may be provided has part of a peri-operative protocol, such as a pre-operative protocol, a post-operative protocol, and/or a sleep protocol.

TABLE 3

| Ingredient | Dosage | Explanation |
|---|---|---|
| Carbohydrate | 800 mL the night | Useful in increasing caloric consumption |
| Rich Drink | before, 400 mL the | before surgery and reducing insulin resistance |
| | day of | after surgery |
| Caloric | See Formulas Above | Ensuring catabolismcachexia or unnecessary |
| Calculations | | weight gain is both important to recovery and |
| | | activity after recovery |
| Essential | 2.5-3 g Leucine, | Some specific essential amino acids can be a |
| Amino | Isoleucine, Valine | more efficient mode of promoting anabolism |
| Acids | | than whole protein |
| Conditionally | Glutamine | During times of high stress, some amino |
| Essential | (10-14 g), | acids, previously non-essential, become |
| Amino Acids | L-alanyl-L- | essential for anabolism and catabolism |
| | glutamine (54 mg | prevention, along with continuation of basic |
| | peptide-N/kg per | functions |
| | day) | |

TABLE 4

| | | |
|---|---|---|
| Timing | Clear liquids 2 | Food 6 hours before surgery, clear liquids 2 |
| | hours before | hours before is safe for the vast majority of |
| | surgery, Nutrition | patients. Carbohydrates before, and beginning |
| | as soon as possible | nutrition as soon as possible after surgery are |
| | after | two of the most important aspects of recovery |
| Omega-3s | 1000-3000 mg | Omega-3s are a natural anti- inflammatory. |
| | Omega 3s before | Immunocompromised patients should be wary |
| | surgery, 1000- | of excessive omega-3 intake |
| | 3000 mg after | |
| Antioxidants | Green Tea | After surgery, oxidative stress is greatly |
| | Extract, Vitamin E | increased, causing side effects like cell |
| | (300-500 mg), | apoptosis |
| | Vitamin C (800- | |
| | 1100 mg) | |

TABLE 5

| | | |
|---|---|---|
| Patient | Before, After | It is important to generate a pain plan and |
| Education | Surgery | tell the patient what procedures are being |
| | | done. This can reduce opioid intake and |
| | | decrease stress |
| Curcumin | 400-600 mg daily | Curcumin is another natural pain reliever |
| | | and anti- inflammatory |
| Ashwagandha | 600 mg or 300 | Natural stress reliever and anti-cortisol |
| root | mg twice daily | ingredient |
| (Withania | | |
| somnifera) | | |
| Lysine and | 2.64 g each daily | Has shown stress relieving qualities in |
| Arginine | | high stress (ie surgery) patients |
| Valerian Root | 300-600 mg before | Valerian root has beneficial effects on |
| (Valeriana | bed | sleep due to its anti-anxiety mechanisms, |
| offcinalis | | working similarly to benzodiazepines, |
| L.) | | binding to the GABA-A receptor and |
| | | decreasing the activity of neurons by |
| | | decreasing the |
| | | influx of choline. |

TABLE 6

| | | |
|---|---|---|
| Tart Cherry | 400-600 mg | Tart cherry supplementation could |
| (Prunus cerasus L) | whenever | improve one's natural sleep cycle, |
| | (preferably | metabolic pathways (specifically |
| | before bed) | melatonin- serotonin pathway) and gut |
| | | health |
| Melatonin | 5-6 mg right | Significant sleep-inducing benefits, with |
| | before bed, just | minimal short-term side effects |
| | a few days after | |
| | surgery | |
| Fiber | 7 g Daily | Fiber can be a good prebiotics for patients |
| | | with bad gut health |
| Chewing Gum | Right after | Chewing gum can help resume normal |
| | surgery, but only | gastric function |
| | if the patient is | |
| | NPO for a | |
| | decent duration | |
| | of time | |
| Fluid and | Carefully | Both considerable negative or positive |
| Electrolyte Balance | manage | fluid and electrolyte balance can be |
| | electrolyte | detrimental to recovery and overall health |
| | and | |
| | fluid balance | |

TABLE 7

| | | |
|---|---|---|
| Music Therapy | Before, during, | Music therapy has been shown to decrease |
| | after surgery | cortisol and stress, and improve outcomes in |
| | | patients |
| Vitamin D | 75-150 mcg | Vitamin D is important to immune, bone, and |
| | Daily | overall health. It is hard to over supplement |
| | | vitamin D, especially during the winter |
| Calcium | 1 g for patients | Calcium is an essential ingredient to bone |
| | with bone | recovery, and calcium needs are increased during |
| | issues | bone repair |

The following include various perioperative embodiments of the invention.

### Daily Pre-Op Protocol.

Embodiments disclosed herein include seven servings of a pre-operative supplemental formula administered over the course of seven days. In some embodiments, each serving comprises a base, glutamine, arginine, Vitamin D, and Ashwagandha. In some embodiments, each serving comprises at least 70 grams, or between 10 and 50 grams, or between 50 and 80 grams, or between 80 and 110 grams of glutamine, at least 21 grams, or between 1 and 10 grams, or between 10 and 30 grams, or between 30 and 45 grams of arginine, 0.001 and 0.003 grams, between 0.003 and 0.020 grams, or between 0.020 and 0.030 grams of Vitamin D, and between 1 and 2 grams, between 2 and 8 grams, or between 8 and 10 grams, or at least 4.2 grams of Ashwagandha. In some embodiments, each serving comprises a base, about 70 grams of glutamine, about 21 grams of arginine, about 0.007 grams of Vitamin D, and about 4.2 grams of Ashwagandha.

A base may include an essential amino acid complex. In some embodiments, a base includes at least five ingredients selected from the following list: L-Histidine, L-Isoleucine, L-Leucine, L-Lysine, L-Methionine, L-Phenylalanine, L-Threonine, L-Valine, and L-Tryptophan. In some embodiments, a base includes L-Histidine, L-Isoleucine, L-Leucine, L-Lysine, L-Methionine, L-Phenylalanine, L-Threonine, L-Valine, and L-Tryptophan.

In some embodiments, a base includes in each serving about 235-245 milligrams of L-Histidine, about 590-620 milligrams of L-Isoleucine, about 2750-2820 milligrams of L-Leucine, about 1040-1080 milligrams of L-Lysine, about 270-295 milligrams of L-Methionine, about 380-420 milligrams of L-Phenylalanine, about 730-770 milligrams of L-Threonine, about 565-595 milligrams of L-Valine, and about 35-45 milligrams of L-Tryptophan. In some embodiments, a base includes in each serving at least five ingredients selected from the following list: about 200-450 milligrams of L-Histidine, about 400-900 milligrams of L-Isoleucine, about 1500-4500 milligrams of L-Leucine, about 1000-2600 milligrams of L-Lysine, about 200-600 milligrams of L-Methionine, about 300-600 milligrams of L-Phenylalanine, about 600-1200 milligrams of L-Threonine, about 400-1100 milligrams of L-Valine, and about 30-80 milligrams of L-Tryptophan.

Embodiments disclosed herein include a method of administering a daily dose of a pre-operative supplement formula for at least seven days prior to surgery.

Table 8 below describes embodiments of a daily pre-operative protocol.

TABLE 8
Daily Pre-Op Protocol

| Nutrients | Reason | How much per day |
|---|---|---|
| Essential | EAAs have been shown to not only provide equal | |
| Amino Acid | muscle recovery as whey protein but also equal | |
| Complex | muscle protein synthesis as whey protein itself. | |
| (EAA's and | EAAs have also been shown to be superior for | |
| BCAA's) | recovery than branched-chain amino acids alone | |
| | (BCAAs) because in the absence of other EAAs, | |
| | BCAAs synthesize and borrow EAA from your | |
| | existing cells. Apart from muscle recovery EAAs | |
| | also play other integral roles within the human | |
| | body and recovery to maintain high-level | |
| | performance. | |
| Sodium | Sodium is both an electrolyte and mineral. It helps | 1-200 mg, 200- |
| | keep the water (the amount of fluid inside and | 500 mg, or 500- |
| | outside the body's cells) and electrolyte balance of | 800 mg |
| | the body. Sodium is also important in how nerves | |
| | and muscles work. | |
| Calcium | Calcium helps building/maintaining bones and | 1-250 mg, 250- |
| | muscle contraction | 550 mg, or 500- |
| | | 850 mg |
| Magnesium | Magnesium is important for many processes in the | 64 milligrams, or |
| | body, including regulating muscle and nerve | about 64 |
| | function, blood sugar levels, and blood pressure | milligrams, or |
| | and making protein, bone, and DNA. | between 64 and 200 |
| | | milligrams |
| Potassium | Helps nerves to function and muscles to contract. | 1-200 mg, 200- |
| | Regulates heartbeat. Helps move nutrients into | 500 mg, or 500- |
| | cells and waste products out of cells. A diet rich | 800 mg, or 800- |
| | in potassium helps to offset some of sodium's | 1000 mg |
| | harmful effects on blood pressure | |
| Glutamine | Glutamine, the most abundant amino acid in the | 1-200 g, 200-500 g, |
| | body, is essential for maintaining intestinal | or 500-800 g |
| | function, immune response, and amino acid | |
| | homeostasis during times of severe stress. | |
| Vitamin D | Vitamin D helps regulate the amount of calcium | 1-100 mcg, or 100- |
| | and phosphate in the body. These nutrients are | 200 mcg, or 200- |
| | needed to keep bones, teeth, and muscles healthy. | 275 mcg |
| Ashwagandha | Ashwagandha is shown to reduce cortisol levels, | 1-200 mg, or, 200- |
| | reduce stress and anxiety, increase muscle | 600 mg, or 600- |
| | recovery and strength, and reduce inflammation. | 80 mg |

Table 9 below discloses embodiments of a daily sleep protocol.

TABLE 9
Daily Sleep Protocol

| | | |
|---|---|---|
| Magnesium | Magnesium plays a role in supporting deep, | 64 milligrams, or |
| | restorative sleep by maintaining healthy levels of | about 64 |
| | GABA, a neurotransmitter that promotes sleep. | milligrams, or |
| | Research indicates supplemental magnesium can | between 64 and 200 |
| | improve sleep quality, especially in people with | milligrams |
| | poor sleep. | |
| Tart Cherry | Data suggests that consumption of a tart cherry | 1-500 mg, or 500- |
| Powder | juice concentrate provides an increase in | 700 mg, or 700- |
| | exogenous melatonin that is beneficial in | 1000 mg |
| | improving sleep duration and quality in healthy | |
| | men and women and might be of benefit in | |
| | managing disturbed sleep. | |
| Valerian Root | Valerian root contains the antioxidants hesperidin | 1-500 mg, or 500- |
| Extract | and linarin, which appear to have sedative | 700 mg, or 700- |
| | and sleep-enhancing properties. Many of these | 1000 mg |
| | compounds may inhibit excessive activity in the | |
| | amygdala, a part of the brain that processes fear | |
| | and strong emotional responses to stress. | |
| L-Theanine | L-theanine helps people fall asleep more quickly | 1-250 mg, 250- |
| | and easily at bedtime, thanks to the relaxation | 550 mg, or 500- |
| | boost it delivers. Research also shows L- | 850 mg |
| | theanine can improve the quality of sleep—not by | |
| | acting as a sedative, but by lowering anxiety and | |
| | promoting relaxation | |
| Lavender | Research has found that lavender increases slow- | 1-30 mg, or 30- |
| Extract | wave sleep, instrumental for slowing heartbeat and | 50 mg, or 50-75 mg |
| | relaxing muscles. | |

Embodiments of a daily post-operative protocol disclosed herein include thirty servings of a post-operative supplement formula. Each serving may include a base, glutamine, Vitamin D, Vitamin C, Vitamin E, Lysine, Iron, Zinc, Vitamin A, and Vitamin K. Each serving may include a base, 1-200 g, 200-500 g, or 500-800 g of glutamine, 0.0-0.1 grams, 0.01-0.08 grams, or 0.08-0.1 grams of Vitamin D, 2-20 grams, 20-40 grams, or 40-60 grams of Vitamin C, 1-5 grams, 5-20 grams, or 20-30 grams of Vitamin E, 1-50 grams, 50-200 grams, or 200-350 grams of Lysine, 0.1-0.2 grams, 0.2-1.2 grams, or 1.2-2 grams of Iron, 0.1-0.2 grams, 0.2-1.2 grams, or 1.2-2 grams of Zinc, 0.01-0.02 grams, 0.020-0.100 grams, or 0.100-0.150 grams of Vitamin A, and 0.0005-0.0010 grams, 0.0010-0.0050 grams, or 0.0050-0.0060 grams of Vitamin K. Each serving may include a base, about 300 grams of glutamine, about 0.03 grams Vitamin D, about 30 grams Vitamin C, about 12 grams Vitamin E, about 90 grams Lysine, about 0.51 grams Iron, about 0.51 grams Zinc, about 0.045 grams Vitamin A, and about 0.0024, grams Vitamin K.

Table 10 below discloses embodiments of a daily post-operative protocol.

TABLE 10
Daily Post-Op Protocol

| | | |
|---|---|---|
| Essential | EAAs have been shown to not only provide equal | |
| Amino Acid | muscle recovery as whey protein but also equal | |
| Complex | muscle protein synthesis as whey protein itself. | |
| (EAA's and | EAAs have also been shown to be superior for | |
| BCAA's) | recovery than branched-chain amino acids alone | |
| | (BCAAs) because in the absence of other EAAs, | |
| | BCAAs synthesize and borrow EAA from your | |
| | existing cells. Apart from muscle recovery EAAs | |
| | also play other integral roles within the human | |
| | body and recovery to maintain high-level | |
| | performance. | |
| Sodium | Sodium is both an electrolyte and mineral. It helps | 1-200 mg, 200- |
| | keep the water (the amount of fluid inside and | 500 mg, or 500- |
| | outside the body's cells) and electrolyte balance of | 800 mg |
| | the body. Sodium is also important in how nerves | |
| | and muscles work. | |
| Calcium | Calcium helps building/maintaining bones and | 1-250 mg, 250- |
| | muscle contraction | 550 mg, or 550- |
| | | 800 mg |
| Magnesium | Magnesium is important for many processes in the | 64 milligrams, or |
| | body, including regulating muscle and nerve | about 64 |
| | function, blood sugar levels, and blood pressure | milligrams, or |
| | and making protein, bone, and DNA. | between 64 and 200 |
| | | milligrams |
| Potassium | It helps your nerves to function and muscles to | 1-200 mg, 200- |
| | contract. It helps your heartbeat stay regular. It | 500 mg, or 500- |
| | also helps move nutrients into cells and waste | 800 mg, or 800- |
| | products out of cells. A diet rich | 1000 mg |
| | in potassium helps to offset some of sodium's | |
| | harmful effects on blood pressure | |
| Glutamine | Numerous studies have shown that | 1-200 g, 200-500 g, |
| | supplementation with glutamine shortens hospital | or 500-800 g |
| | stays; decreases infection rates, improves overall | |
| | nitrogen balance in patients and enhances net | |
| | protein synthesis, especially for skeletal muscle. | |
| Vitamin D | Vitamin D is essential for bone healing and | 1-100 mcg, or 100- |
| | muscle function and is critical for a patient's | 200 mcg, or 200- |
| | recovery | 275 mcg |
| Vitamin C | Vitamin C requirement is increased | 100-300 mg, or 300- |
| | in surgical patients, and the potential advantage of | 500 mg, or 500- |
| | supplementation is to increase the plasma and | 700 mg |
| | tissue levels of vitamin C and thereby reduce | |
| | oxidative stress. | |
| Vitamin E | Anecdotal reports claim that vitamin E speeds | 1-400 mg, or 400- |
| | wound healing and improves the cosmetic | 700 mg, or 700- |
| | outcome of incisions and other wounds. It also has | 1000 mg |
| | antioxidant/disease fighting properties. | |
| Lysine | Lysine can improve wound healing in your body. | 2.0-2.64 g, or 2.64- |
| | Lysine is required for the formation of collagen, a | 3 g, or 3-5 g |
| | protein that acts as a scaffold and helps support | |
| | and give structure to skin and bones. | |
| Iron | Ideal for forming hemoglobin and carrying | 0.1-0.2 g, 0.2-1.2 g, |
| | oxygen. | or 1.2-2 g |
| Zinc | Zinc is an essential trace mineral for DNA | 0.1-0.2 g, 0.2-1.2 g, |
| | synthesis, cell division, collagen formation, | or 1.2-2 g |
| | protein synthesis, and immune function - all | |
| | necessary processes for tissue regeneration and | |
| | wound repair. Zinc is necessary to develop and | |
| | activate T-lymphocytes, which are important for | |
| | the immune system. | |
| Vitamin A | This vitamin supports adequate healing of | 0.01-0.02 g, 0.020- |
| | incisions and promotes collagen | 0.100 g, or 0.100- |
| | production. Vitamin A may increase the tensile | 0.150 g |
| | strength of the healing incision, which is a good | |
| | thing! It does this by prompting the inflammatory | |
| | response to the wound. | |
| Vitamin K | Vitamin K is an important vitamin in postsurgical | 0.0005-0.0010 g, or |
| | healing due to its blood clotting activity. It | 0.0010-0.0050 g, or |
| | promotes blood clotting in the body and reduces | 0.0050-0.0060 g |
| | the recovery duration. Vit K can also be used after | |
| | surgery to prevent incisions and any bleeding | |
| | complications effectively. | |

In some embodiments, a Pre- and Post-operative OMEGA Protocol is provided. Table 11 below discloses embodiments of a pre- and post-operative OMEGA protocol.

TABLE 11
Daily Pre- and Post-Op OMEGA Protocol

| | | |
|---|---|---|
| OMEGA | In the medical community today, there's | 2000 mg |
| | excitement that omega-3 fatty acids may actually | |
| | reduce the risk of infections and complications | |
| | post-surgery. In addition, studies indicate that | |
| | omega-3s could potentially improve microbiome | |
| | health, regulate constipation, ease anxiety, and | |
| | reduce the need for excessive painkillers - all | |
| | common concerns for surgical patients. | |

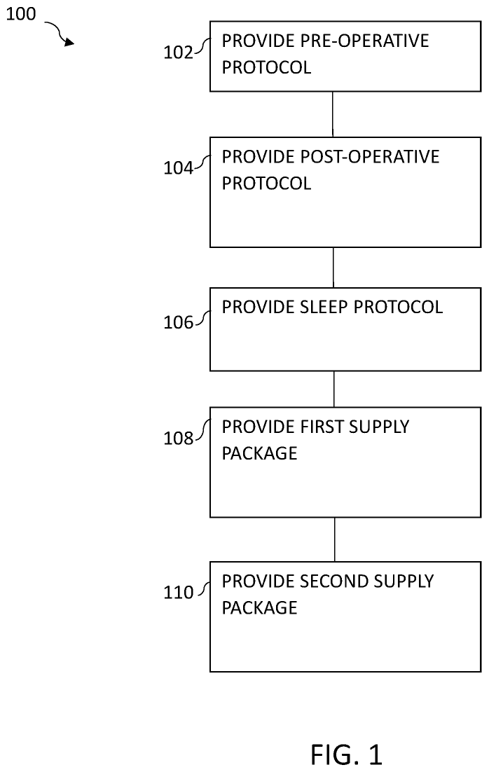

Turning now to FIG. 1, some embodiments herein include a method of improving patient recovery after a surgical procedure, the method 100 including one or more of providing a pre-operative protocol 102; providing a post-operative protocol 104; and/or providing a sleep protocol 106.

Providing a pre-operative protocol 102 may include providing a pre-operative protocol having a first set of instructions for the consumption of a first nutritional supplement.

Providing a post-operative protocol 104 may include providing a post-operative protocol having a second set of instructions for the consumption of a second nutritional supplement different from the first nutritional supplement.

The first set of instructions may include an instruction to at least one of consume or administer the pre-operative protocol for seven days prior to the surgery. The first nutritional supplement may include a base, vitamin D, Calcium, magnesium, potassium, sodium, ashwagandha, glutamine, and arginine in quantities selected to improve recovery after surgery. The base may be a base as previously described herein.

The second set of instructions may include an instruction to at least one of consume or administer the post-operative protocol for thirty days after surgery. The second nutritional supplement may include a base, vitamin D, Calcium, magnesium, potassium, sodium, ashwagandha, glutamine, and arginine in quantities selected to improve recovery after surgery. The base may be a base as previously described herein.

In some embodiments, each serving of the first nutritional supplement includes: a base; glutamine; arginine; Vitamin D; and Ashwagandha. Each serving of the first nutritional supplement may include 100 micrograms Vitamin D; 250 milligrams Calcium; 175 milligrams Magnesium; 500 milligrams Potassium; and 200 milligrams Sodium. Each serving of the first nutritional supplement may include between 10 and 12 grams glutamine; between 10 and 30 grams arginine; and between 200 and 600 milligrams Ashwagandha.

The first set of instructions may include an instruction to consume one serving a day of the first nutritional supplement for seven days prior to the surgery.

The second set of instructions may include at least one of: an instruction to consume one serving a day of the second nutritional supplement for thirty days after the surgery; or an instruction to consume one serving a day of the second nutritional supplement for sixty days after the surgery. The second set of instructions may include an instruction to consume one serving a day of the second nutritional supplement for at least thirty days after the surgery. The second set of instructions may include an instruction to consume one serving a day of the second nutritional supplement for between least thirty and sixty days after the surgery.

Continuing with FIG. 1, the second nutritional may include leucine; isoleucine; and valine.

In some embodiments, the method 100 further comprises providing a sleep protocol 106, the sleep protocol having a third set of instructions for the consumption of a third nutritional supplement different from the first and second nutritional supplements, the third nutritional supplement having tart cherry powder, valerian root extract, L-theanine, and lavender extract, the third set of instructions further comprising instructions to consume the third nutritional supplement for a period of time.

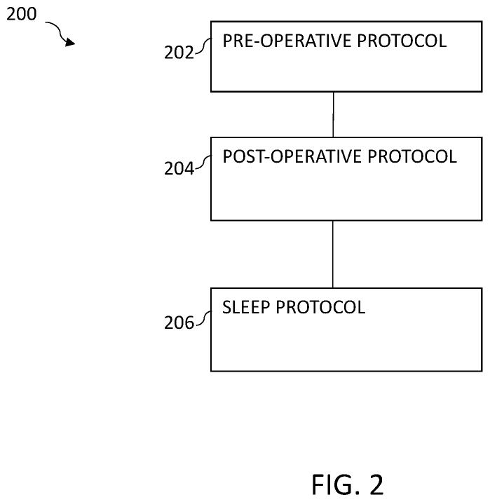

Turning now to FIG. 2, a perioperative protocol 200 is described. The peri-operative protocol 200 may include a pre-operative protocol 202 as described herein, a post-operative protocol 204 as described herein, and, optionally, a sleep protocol 206 as described herein.

Embodiments herein may include one or more nutritional supplements described herein packaged with instructions for use in a peri-operative protocol, a pre-operative protocol, and/or a sleep protocol.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, and as an example, the disclosure of a “packaging” should be understood to encompass disclosure of the act of “packaging”-whether explicitly discussed or not- and, conversely, were there only disclosure of the act of “packaging”, such a disclosure should be understood to encompass disclosure of a “packaging mechanism”. Such changes and alternative terms are to be understood to be explicitly included in the description.

Moreover, the claims shall be construed such that a claim that recites “at least one of A, B, or C” shall read on a device that requires “A” only. The claim shall also read on a device that requires “B” only. The claim shall also read on a device that requires “C” only.

Similarly, the claim shall also read on a device that requires “A+B”. The claim shall also read on a device that requires “A+B+C”, and so forth.

The claims shall also be construed such that any relational language (e.g. perpendicular, straight, parallel, flat, etc.) is understood to include the recitation “within a reasonable manufacturing tolerance at the time the device is manufactured or at the time of the invention, whichever manufacturing tolerance is greater”.

Those skilled in the art can readily recognize that numerous variations and substitutions may be made in the invention, its use, and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

What is claimed is:

1. A method of improving patient recovery after a surgery, the method comprising:
providing a pre-operative protocol, the pre-operative protocol having a first set of instructions for the consumption of a first nutritional supplement; and
providing a post-operative protocol, the post-operative protocol having a second set of instructions for the consumption of a second nutritional supplement different from the first nutritional supplement; wherein
the first set of instructions comprises an instruction to at least one of consume or administer the pre-operative protocol for seven days prior to the surgery;
the first nutritional supplement comprises a base, vitamin D, Calcium, magnesium, potassium, sodium, ashwagandha, glutamine, and arginine in quantities selected to improve recovery after surgery;
the second set of instructions comprises an instruction to at least one of consume or administer the post-operative protocol for thirty days after surgery; and
the second nutritional supplement comprises a base, vitamin D, Calcium, magnesium, potassium, sodium, ashwagandha, glutamine, and arginine in quantities selected to improve recovery after surgery.

2. The method of claim 1, wherein each serving of the first nutritional supplement comprises:
a base;
glutamine;
arginine;
Vitamin D; and
Ashwagandha.

3. The method of claim 2, wherein each serving of the first nutritional supplement comprises:
100 micrograms Vitamin D;
250 milligrams Calcium;
175 milligrams Magnesium;
500 milligrams Potassium; and
200 milligrams Sodium.

4. The method of claim 2, wherein each serving of the first nutritional supplement comprises:
between 10 and 12 grams glutamine;
between 10 and 30 grams arginine; and
between 200 and 600 milligrams Ashwagandha.

5. The method of claim 1, wherein the first set of instructions comprises an instruction to consume one serving a day of the first nutritional supplement for seven days prior to the surgery.

6. The method of claim 1, wherein the second set of instructions comprises at least one of:
an instruction to consume one serving a day of the second nutritional supplement for thirty days after the surgery; or
an instruction to consume one serving a day of the second nutritional supplement for sixty days after the surgery.

7. The method of claim 1, wherein the second set of instructions comprises:
an instruction to consume one serving a day of the second nutritional supplement for at least thirty days after the surgery.

8. The method of claim 1, wherein the second set of instructions comprises:
an instruction to consume one serving a day of the second nutritional supplement for between least thirty and sixty days after the surgery.

9. The method of claim 1, wherein the second nutritional supplement comprises:
leucine;
isoleucine; and
valine.

10. The method of claim 1, further comprising:
providing a sleep protocol, the sleep protocol having a third set of instructions for the consumption of a third nutritional supplement different from the first and second nutritional supplements, the third nutritional supplement having tart cherry powder, valerian root extract, L-theanine, and lavender extract, the third set of instructions further comprising instructions to consume the third nutritional supplement for a period of time.

11. The method of claim 1, further comprising:
providing the pre-operative protocol and seven servings of the first nutritional supplement in a first supply package; and
providing the post-operative protocol and at least thirty servings of the second nutritional supplement in a second supply package.

12. A perioperative protocol, comprising:
a pre-operative protocol, the pre-operative protocol having a first set of instructions for the consumption of a first nutritional supplement; and
a post-operative protocol, the post-operative protocol having a second set of instructions for the consumption of a second nutritional supplement different from the first nutritional supplement; wherein
the first set of instructions comprises an instruction to at least one of consume or administer the pre-operative protocol for seven days prior to the surgery;
the first nutritional supplement comprises a base, vitamin D, Calcium, magnesium, potassium, sodium, ashwagandha, glutamine, and arginine in quantities selected to improve recovery after surgery;
the second set of instructions comprises an instruction to at least one of consume or administer the post-operative protocol for thirty days after surgery; and
the second nutritional supplement comprises a base, vitamin D, Calcium, magnesium, potassium, sodium, ashwagandha, glutamine, and arginine in quantities selected to improve recovery after surgery.

13. The perioperative protocol of claim 12, wherein each serving of the first nutritional supplement comprises:
a base;
glutamine;
arginine;
Vitamin D; and
Ashwagandha.

14. The perioperative protocol of claim 13, wherein each serving of the first nutritional supplement comprises:
100 micrograms Vitamin D;
250 milligrams Calcium;
175 milligrams Magnesium;
500 milligrams Potassium; and
200 milligrams Sodium.

15. The perioperative protocol of claim 13, wherein each serving of the first nutritional supplement comprises:
between 10 and 12 grams glutamine;
between 10 and 30 grams arginine; and
between 200 and 600 milligrams Ashwagandha.

16. The perioperative protocol of claim 12, wherein the first set of instructions comprises an instruction to consume one serving a day of the first nutritional supplement for seven days prior to the surgery.

17. The perioperative protocol of claim 12, wherein the second set of instructions comprises at least one of:
an instruction to consume one serving a day of the second nutritional supplement for thirty days after the surgery; or
an instruction to consume one serving a day of the second nutritional supplement for sixty days after the surgery.

18. The perioperative protocol of claim 12, wherein the second set of instructions comprises:
an instruction to consume one serving a day of the second nutritional supplement for at least thirty days after the surgery.

19. The perioperative protocol of claim 12, wherein the second set of instructions comprises:
an instruction to consume one serving a day of the second nutritional supplement for between least thirty and sixty days after the surgery.

20. The perioperative protocol of claim 12, wherein the second nutritional supplement comprises:
leucine;
isoleucine; and
valine.

21. The perioperative protocol of claim 12, further comprising:
providing a sleep protocol, the sleep protocol having a third set of instructions for the consumption of a third nutritional supplement different from the first and second nutritional supplements, the third nutritional supplement having tart cherry powder, valerian root extract, L-theanine, and lavender extract, the third set of instructions further comprising instructions to consume the third nutritional supplement for a period of time.
